(19) United States
(12) Patent Application Publication
(10) Pub. No.: US 2016/0030229 A1
(43) Pub. Date: Feb. 4, 2016

(54) METHOD AND SYSTEM FOR BREATHING MODULATION

(71) Applicant: GE SLEEPING TECHNOLOGIES LTD., Haifa (IL)

(72) Inventors: Emmanuel Goldschmidt, Haifa (IL); Gilad Ben Tzur, Gan Ner (IL)

(21) Appl. No.: 14/774,778

(22) PCT Filed: Mar. 13, 2014
(86) PCT No.: PCT/IL2014/050270

§ 371 Date: Sep. 11, 2015

Related U.S. Application Data
(60) Provisional application No. 61/778,439, filed on Mar. 13, 2013.

(51) Int. Cl. A61F 5/56; A61M 16/06; A61M 16/20; A61M 16/00

(52) U.S. Cl. CPC A61F 5/56; A61M 16/0057; A61M 16/0666; A61M 16/0688; A61M 16/20

(57) ABSTRACT

A nasal breathing aid comprising the following components: a housing; facial engagement means attached thereto; at least two nostril pieces coupled to the housing. On the housing, there are at least two nasal inlets; at least one vent; and in the housing: a first passage and a second passage, each communicating an inlet with a vent, and fluid-isolated from each other, and restriction means configured to allow free flow of air through each passage from a vent to an inlet, and flow of air through the first passage independently restricted from flow of air through the second passage, from an inlet to a vent.

### FIELD OF THE INVENTION

The present invention relates to a system and method for modulation of breathing in individuals suffering from disorders such as apnea

### BACKGROUND OF THE INVENTION

Sleep disorders such as apnea can adversely affect health and social life.

It is estimated that in the U.S. the average untreated sleep apnea patient's annual health care costs $1,336 more than an individual without sleep apnea. This may cause $3.4 billion/year in additional medical costs.

Although some devices and methods have been used for treatment of apnea, the vast majority of the treated individuals have found the treatment to be uncomfortable or inconvenient and thus discontinue the treatment.

One objective is to provide a system and treatment that are more convenient to use.

WO2009117400 describes methods of adjusting the resistance of a nasal device.

Another objective is providing improved systems and methods for adjusting the resistance.

### SUMMARY OF THE INVENTION

According to one aspect, a system is provided that allows continuous and prolonged air regulation, i.e. control of breathing through the nostrils so as to reduce or eliminate apnea, without requiring tubes, air blowing motors, pump and other equipment that hinder movement during sleep and might be unhealthy due to harbor of pathogens etc.

According to another aspect, a system is provided that allows separate control of breathing through each nostril.

Such system may include tubes, pump and other equipment, but have hardware to separately control breathing via each nostril.
A nasal breathing aid is provided, comprising the following components:
Housing;
Facial engagement means attached thereto;
At least two nostril pieces coupled to the housing;
On the housing:
   At least two nasal inlets;
   At least one vent;
In the housing:
   A first passage and a second passage, each communicating an inlet with a vent, and fluid-isolated from each other, and
Restriction means configured to allow:
   Free flow of air through each passage from a vent to an inlet, and flow of air through the first passage independently restricted from flow of air through the second passage, from an inlet to a vent.
The aid in some embodiments further comprises means to alternately adjust restriction of air through the first passage and the second passage.
The aid may further comprise means to adjust restriction of air through the first passage and the second passage to different extents.
According to one aspect, a nasal breathing aid is provided, comprising the following components:
Housing;
Facial engagement means attached thereto;
At least two nostril pieces coupled to the housing;
On the housing:
   At least two nasal inlets;
   At least one vent;
In the housing;
   At least one passage communicating the inlets with the vents, and
Restriction means configured to allow:
   Unimpeded flow of air through the passage from the vents to the inlets, and
   Restricted flow of air through the passage from the inlets to the vents, and
Characterized by:
   The breathing aid excluding skin-adhesive material; The aid being wearable;
   Restriction means comprising:
      membranes, each comprising an outer perimeter and an inner perimeter, the inner perimeter defining an orifice; wherein the outer perimeter is engaged between a nostril piece and the housing; and wherein the inner perimeter abuts the inlet when air is exhaled through the nostril pieces and does not abut the inlet, and the orifice expands towards the nostril pieces, when air is inhaled through the nostril pieces.
The breathing aid of claim may further comprise at least one resilient valve, each conforming to an inlet such that below a predetermined the valve essentially blocks the inlet, and at a predetermined exhale force the valve unblocks the inlet.
In some embodiments the exhale valve comprises an orifice with an adjustable diameter, being operated via hydraulic or pneumatic power.
The inside diameter of the orifice, or the outside, are made as a hollow ring-shaped tube that has an opening to enable its inflation by hydraulic or pneumatic power.
The aid may further comprise an actuator configured to allow adjusting a force holding the valve against the inlet.
Some embodiments further comprise:
An elastic strap coupled to the housing;
Electric wiring within or on the strap;
Automatic control means programmed to control the actuator, wherein the electrical wiring electrically connects the automatic control means with the actuator.
In some embodiments the breathing aid further comprises swivel shutters, static cages and at least one screw drive, wherein the screw drive is coupled to the swivel shutters, and the swivel shutters are arranged relative to the cages such that rotation of the screw drives rotates at least one shutter, to adjust gaps between the cages and the shutters.
Some embodiments further comprise at least one dial protruding from the housing, the dials coupled to the screw drives such that manipulation of a dial leads to rotation of at least one shutter.
Some embodiments further comprise easily replaceable mucus-absorbent pads.
In some embodiments the pads are disposed in the nostril pieces.
In some embodiments the pads are disposed in the passages.
Each pad may comprise:
An essentially flat layer of mucus-absorbent material;
Ribs attached to the layer,
And a handle extending from the ribs.
The valves are spring loaded in some embodiments.
The valve may comprise an elastomeric element proximal to the inlet such compression of the element impedes flow of air through the passages.
Some embodiments further comprise at least one air pump and tubing coupling the pump to the housing.
In some embodiments the aid is configured to allow the pump to alternately supply air to each nasal inlet.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1a depicts one breath modulator embodiment attachable with an elastic strap and including a valve that allows free entry of air into the modulator but restricts passage of air thereout.

FIG. 1b shows another embodiment, having a slightly different structure. The elastic strap includes therein or thereon wiring that is electrically connectable to a computer, or a hard-wired relay system or other means that is configured to allow programmed control of the valve.

FIG. 1c demonstrates another embodiment wherein there are two valves, one for each nostril. Each valve may be individually controlled, by either manipulation of a manual control or of a motor inside the modulator.

FIG. 2 shows a cross-section of a portion from modulator. The modulator includes controller, a nostril piece, a membrane, a cone-shaped inlet, casing, spring loaded ball valve, air vents and actuator.

FIGS. 3a and 3b depicts the operation of the latter modulator.

FIG. 4 illustrates another section of a modulator, showing ball valves. The resistance of the valves is controlled with a screw.

FIG. 5a shows in two perspective views another embodiment having nostril pieces.

FIG. 5b shows the same in a different perspective view. 5 B

FIG. 6a shows sections of the latter modulator in perspective view from various angles. The valves include membrane (only one shown for clarity), swivel shutters, static cages, and a screw drive.

The embodiment is further shown in FIG. 6b.

FIG. 7 depicts a similar modulator that includes non-woven disposable pads that may be used to absorb mucus secreted from nostrils.

FIG. 8 shows a similar embodiment, with an installed and disposable pad.

FIG. 9 shows the embodiment depicted in FIG. 8, with the pad removed.

FIG. 10 depicts a valve system in some other embodiments, in which an actuator, either mechanical or electrical, applies pressure upon a flexible article to restrict or ease flow of exhaled air.

FIG. 11 shows a flexible ring that bends inwards, under the pressure of a disk, thus narrowing an air orifice.

In FIG. 12 a cutout of a modulator controlled by an electromechanical mechanism is shown. Note that breathing via nostrils is separately controlled for each nostril.

FIG. 13 shows a similar system, however the valve control mechanism is entirely mechanical.

FIG. 14 illustrates a system similar to commercially available systems, i.e. which includes a pump (not shown) and tubing; however, the embodiment provides separate supply of air to each nostril.

FIG. 15a depicts an exploded perspective view of another embodiment. The modulator comprises a V-shaped valve. The valve conforms in shape to an interior wall of the modulator, which has vents therealong.

As shown in FIG. 15b, the modulator further comprises swivel shutters, and static cages.

FIG. 16a shows another embodiment. The modulator again has vents on its casing; a knob is operationally connected to a thread, and the thread is coupled to a shutter with slits via a gear.

FIG. 16b further shows the embodiment depicted in FIG. 16a.

FIG. 17a illustrates in perspective views another modulator in which there is an article in each air passage allowing control of the resistance to exhalation. The article comprises pores that are blocked or distorted as a result of being squeezed by closing screws.

FIG. 17b depicts the modulator shown in FIG. 17a, in cross sectional view.

FIG. 18a depicts a perspective view of another article which is flexible, and which comprises an orifice.

As shown in FIG. 18b, in comparison to FIG. 18a, applying pneumatic or hydraulic pressure in the article leads to the orifice diminishing in size.

FIG. 18c illustrates a cross-section of the article as shown in FIG. 18a.

FIG. 18d illustrates a cross-section of the article as shown in FIG. 18b.

FIG. 19a schematically presents an embodiment that can modulate the breathing. The embodiment comprises a motor, a timer and a microprocessor.

FIG. 19b schematically presents a second embodiment comprising a motor, a microprocessor and a sensor.

FIG. 19c schematically presents a third embodiment comprising two motors, a microprocessor and two sensors.

### DESCRIPTION OF THE DRAWINGS AND EXEMPLARY EMBODIMENTS OF THE INVENTION

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The terms “comprises”, “comprising”, “includes”, “including”, and “having” together with their conjugates mean “including but not limited to”.

The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

In discussion of the various figures described herein below, like numbers refer to like parts. The drawings are generally not to scale. For clarity, non-essential elements were omitted from some of the drawings. Some optional elements are marked by dashed lines.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

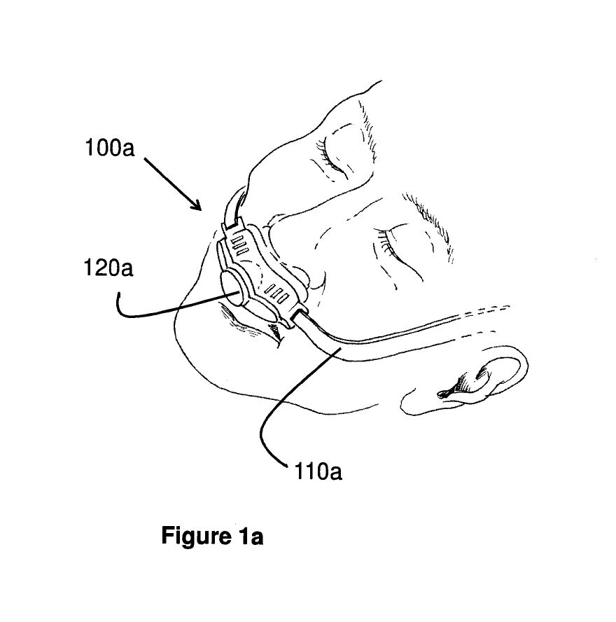

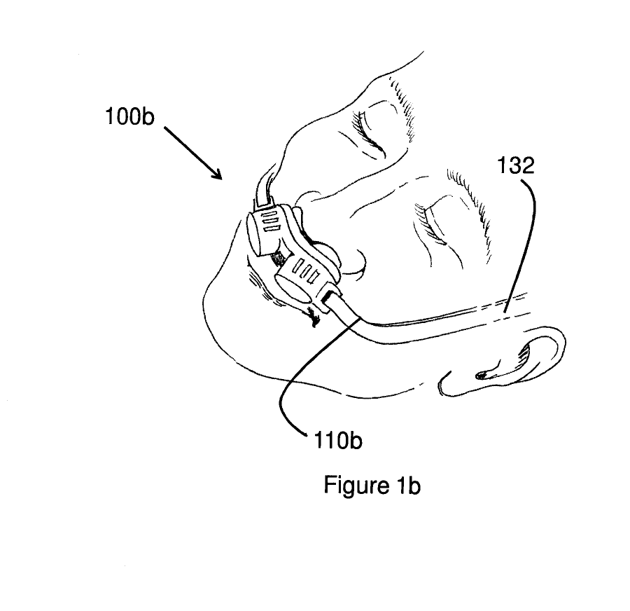

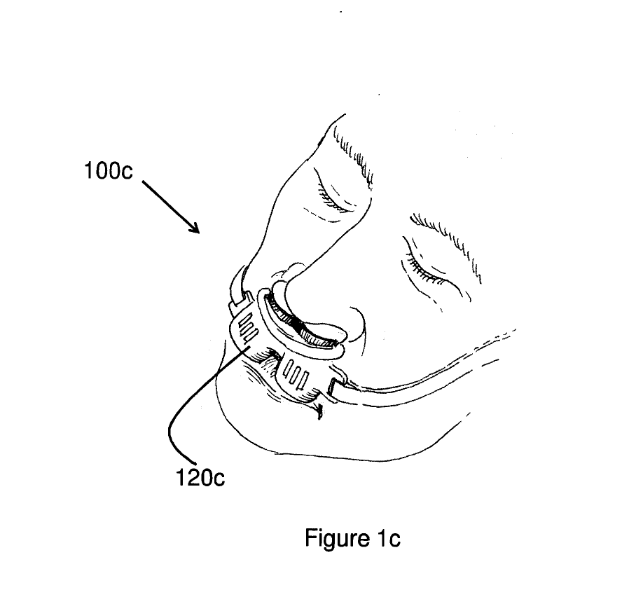

FIG. 1a depicts one breathing modulator embodiment 100a. The modulator 100a is simply attachable with an elastic strap 110a. The modulator 100a includes a valve 120a that allows free entry of air into the modulator 100a but restricts passage of air thereout. Note that counter to commercially available sleep modulators, there are no parts that can fall off during sleep, or attached to the modulator 100a by a wire or tube that limit the user's movement. Such “external” parts would irritatingly have to be carried with the user or disconnected, when waking up and wishing to go to the restroom in the middle of the night for example.

In general, the valve/s may be controlled by motor/s within the modulator or mechanically controlled, as will be explained and shown below. Typically, the embodiment 100a shown in the figure is mechanically controlled.

FIG. 1b shows another embodiment 100b, having a slightly different structure. The elastic strap 110b includes therein or thereon wiring 132 that is electrically connectable to a computer, or a hard-wired relay system or other means that is configured to allow programmed control of the valve as will be explained below.

FIG. 1c demonstrates another embodiment 100c wherein there are two valves 120c, one for each nostril. Each valve may be individually controlled, by either manipulation of a manual control or of a motor inside the modulator 100c.

The control of the valves such as valves 120c may be programmed to simultaneously and/or symmetrically provide each valve with the same instructions, for example that the valve shut or partially close/open, or may differ, for example stagger the operation of the valves, such that one valve at a time is shut, or close/open the valves to a different extent/period of time. Such staggered or individual operation may help when one nasal passage is blocked or different from the other passage, or when training a user in usage of the modulator. Indeed, it has been discovered that a major obstacle to prolonged use of the commercially available modulators is their effect on the breathing pattern, as the change is predominantly uncomfortable. Some of the embodiments thus allow gradually increasing and/or changing the modulation. This adjustment can be programmed or mechanically controlled. The adjustment also serves to customize the resistance to the particular needs of various individuals.

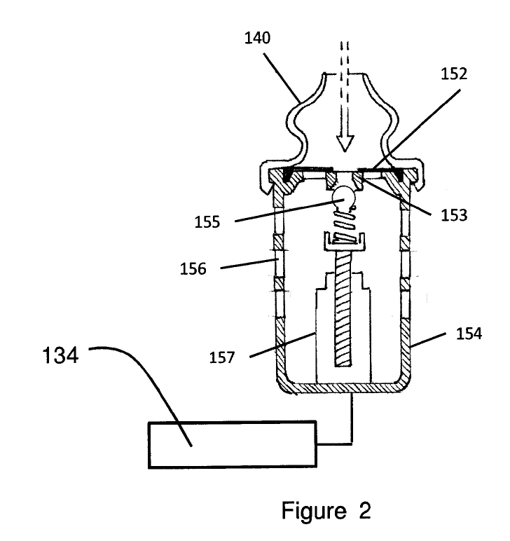

FIG. 2 shows a cross-section of a portion from modulator 100c. The modulator 100c includes controller 134, a nostril piece 140, a membrane 152, a cone-shaped inlet 153, casing 154, spring loaded ball valve 155, air vents 156 and actuator 157.

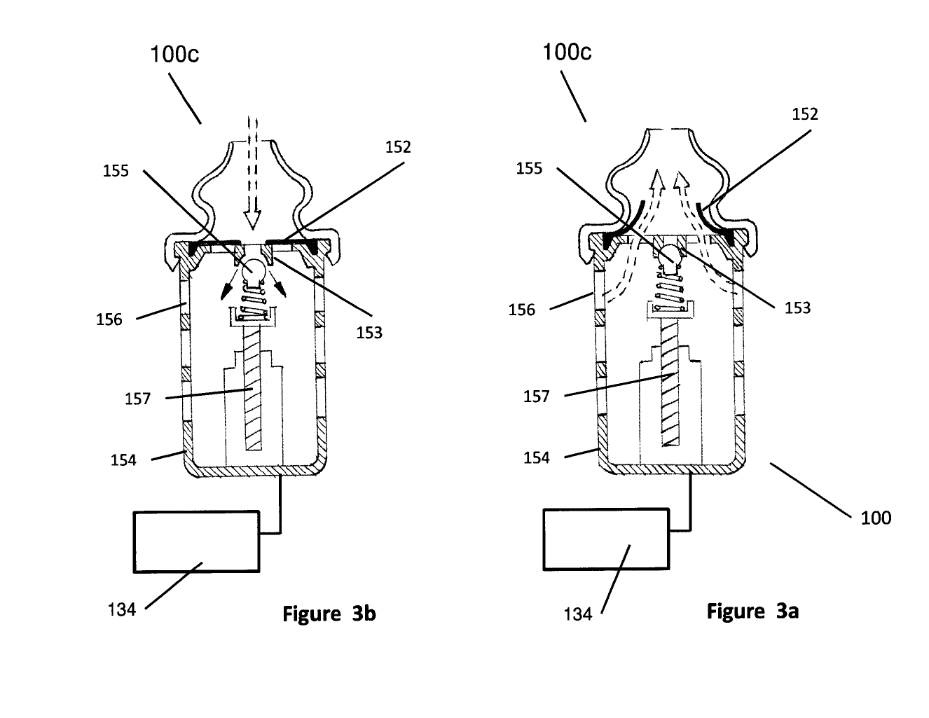

Operation of the modulator 100c is shown in FIGS. 3a and 3b.

The breathing aid 100c comprises a resilient valve 115 conforming to the inlet such that below a predetermined exhale force the valve essentially blocks the inlet, and at a predetermined exhale force the valve unblocks the inlet.

Membrane 152 is configured to serve as a check valve: Air is freely accessible via vents 156, and membrane 152 that opens. However, when air is exhaled, the membrane 152 is restored to a flat closed shape and thus a pressure builds inside a nostril that acts to reduce snoring. Exhalation is achieved by build up of exhale force that suffices to move the ball valve 155 backwards, as shown in FIG. 3b, that allows air to escape via vents 156.

In some embodiments the exhale valve comprises an orifice with an adjustable diameter, being operated via hydraulic or pneumatic power. The inside diameter of the orifice, or the outside, are made as a hollow ring-shaped tube that has an opening to enable its inflation by hydraulic or pneumatic power.

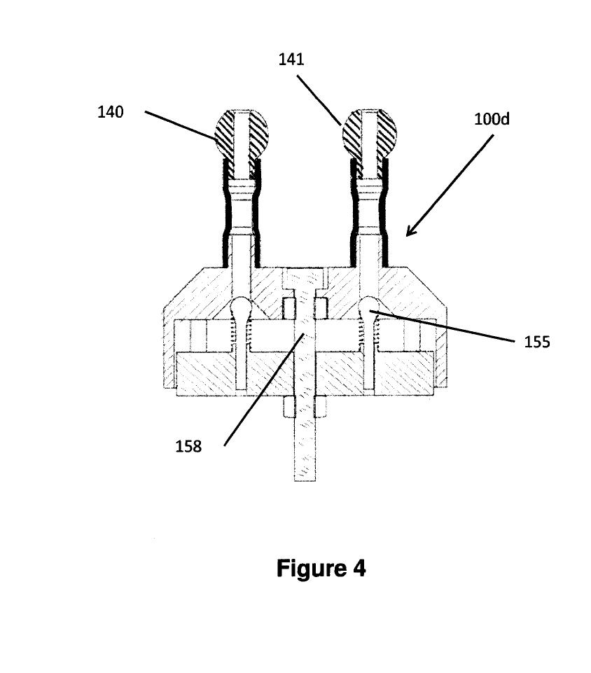

FIG. 4 illustrates another section of a modulator 100d, showing ball valves 155′. The resistance of the valves is controlled with a screw 158. The spherical form 141 of the nostril pieces 140 act as a sealing improvement for the nostrils and also as nostrils airway openers for easier inhalation.

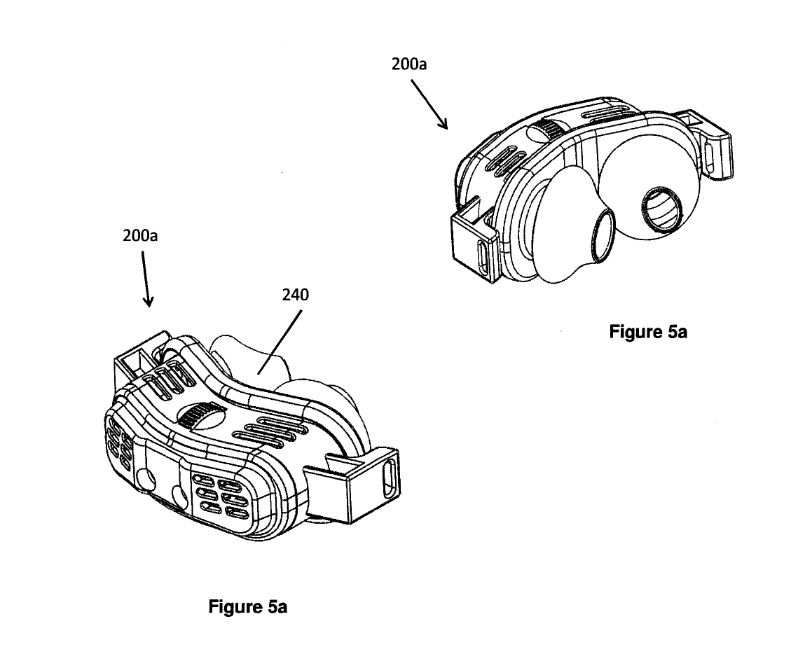

FIGS. 5a and 5b show in two perspective views another embodiment 200a having nostril pieces 240.

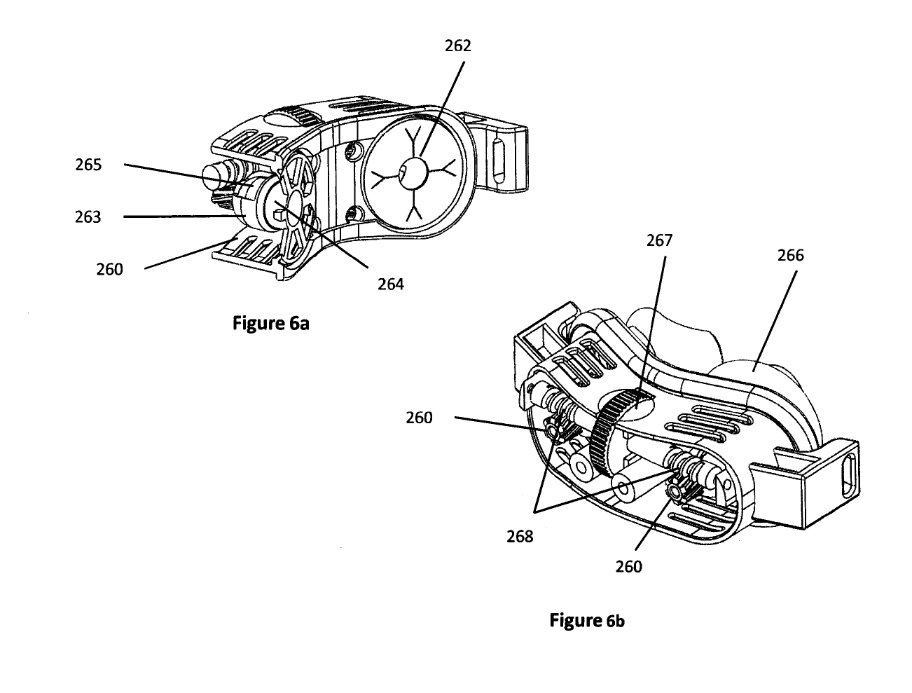

FIGS. 6a, 6b show sections of the modulator 200a in perspective view from various angles. The valves 260 include:

membrane 262 (only one shown for clarity), swivel shutters 263, static cages 264, and a screw drive 266. The screw drive includes a wheel 267 that is manually controlled from outside the modulator 200a, and a double thread 268 connected thereof and to the swivel shutters 263. Manipulation of the wheel 267 leads to rotation of coupled shutters 263, to adjust gaps 265 between the shutters 263 and the cages 264, thereby it adjusting the resistance of the valves 260 to exhaled air.

In other embodiments, there are two wheels, each controlling a swivel shutter, to allow separate control of each valve.

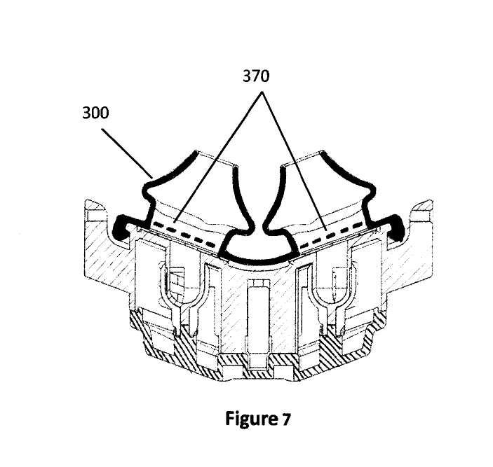

FIG. 7 depicts a similar modulator 300 that includes non-woven disposable pads 370 that may be used to absorb mucus secreted from nostrils.

FIG. 8 shows a similar embodiment 300′, with an installed and disposable pad 370′.

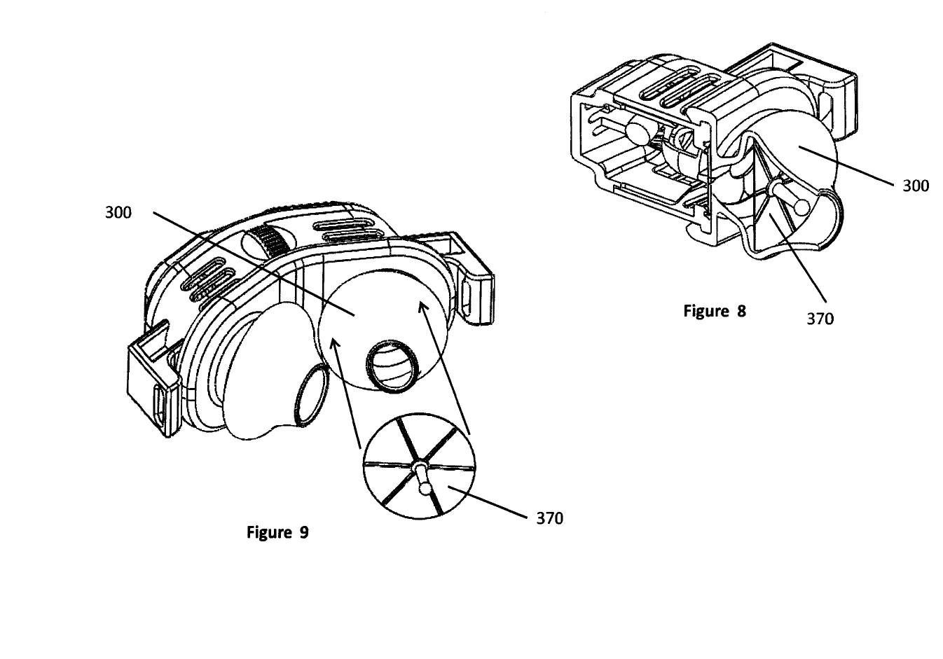

FIG. 9 shows the embodiment 300′ depicted in FIG. 8, with the pad 370′ removed.

In some embodiments the exhale valve comprises an orifice with an adjustable diameter, being operated via hydraulic or pneumatic power. The inside diameter of the orifice, or the outside, are made as a hollow ring-shaped tube that has an opening to enable its inflation by hydraulic or pneumatic power.

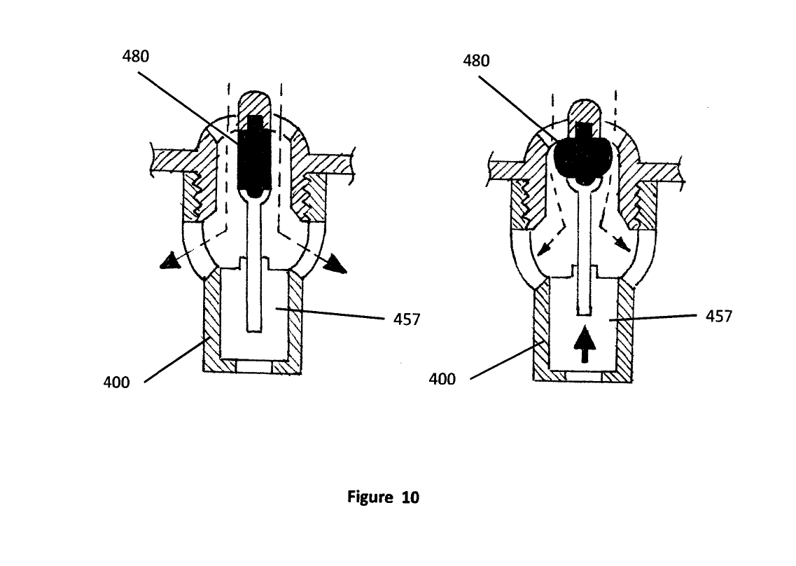

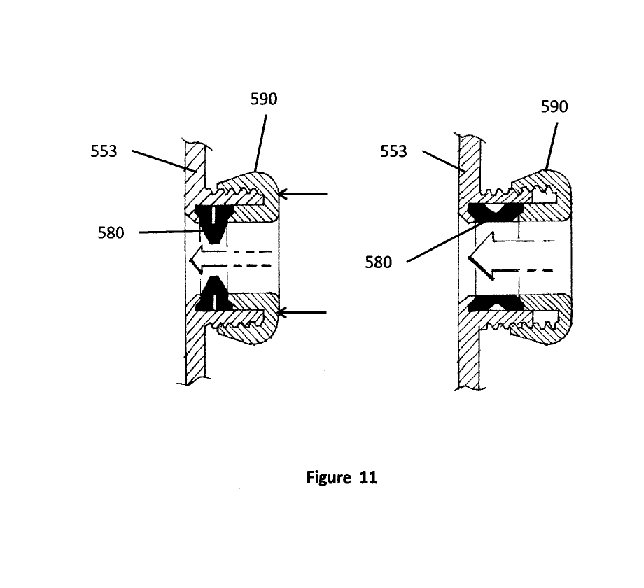

FIG. 10 and FIG. 11 depict a valve system in some other embodiments 400 and 500 respectively, in which an actuator 457 (not shown in FIG. 11), either mechanical or electrical, applies pressure upon a flexible article 480, 580 to restrict or ease flow of exhaled air. Element 480 is an elastomeric material that changes its diameter according to the pressure applied by the actuator 457 and thus changes the exhale flow resistance.

The actuator 457 in the embodiment 400 shown in FIG. 10 acts somewhat like the actuator 157 in the embodiment 100 depicted in FIG. 2. The actuator in the embodiment 500 shown in FIG. 11 threads a threaded knob 590 onto an inlet 553 with a matching thread.

In FIG. 12 a cutout of a modulator 600 controlled by an electromechanical mechanism 690 is shown. Note that breathing via nostrils is separately controlled for each nostril.

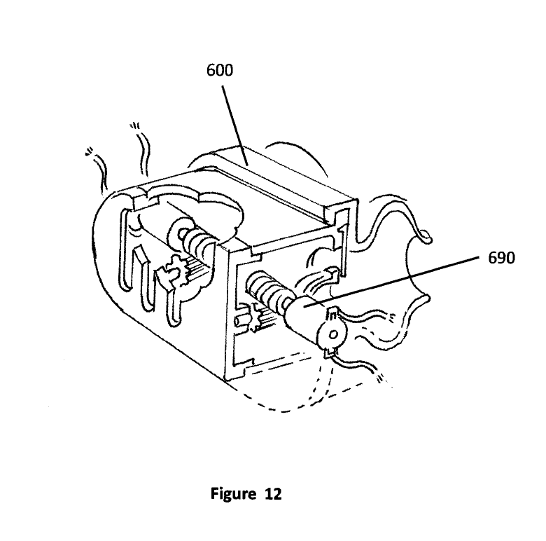

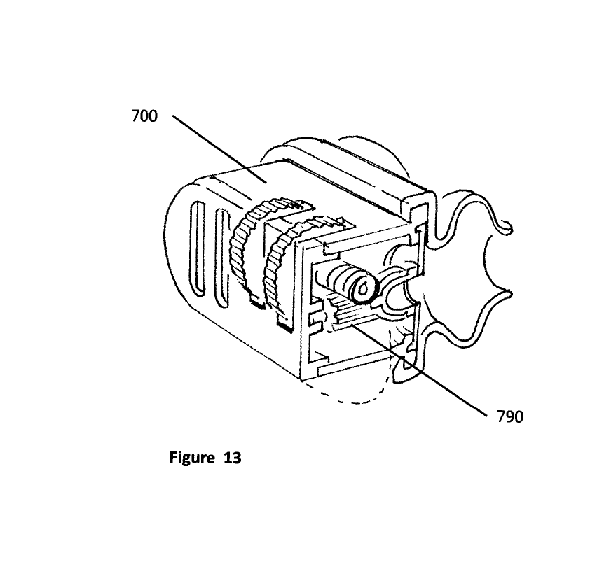

FIG. 13 shows a similar system 700; however the valve control mechanism 790 is entirely mechanical.

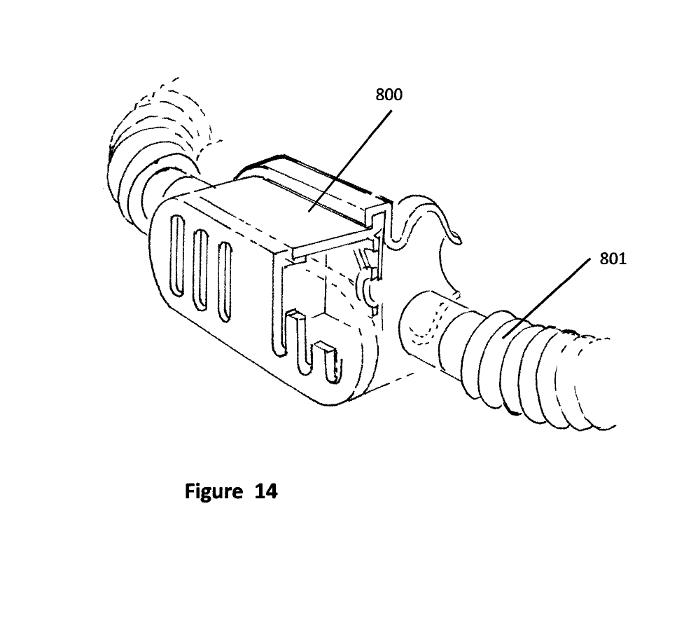

FIG. 14 illustrates a system 800 similar to commercially available systems, i.e. which includes a pump (not shown) and tubing 801; however, the embodiment 800 provides separate supply of air to each nostril. In some embodiments each supply is provided by a separate pump, or in others a single pump is set to alternate between the valves for each nostril. Such alternation would be controlled by a component such as a programmed processor. Other embodiments have filter pads, configured to filter inhaled air.

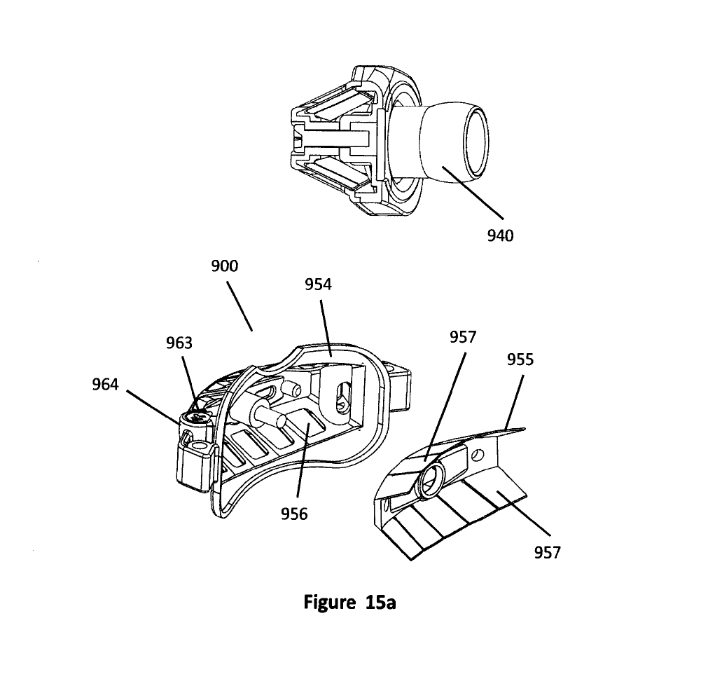

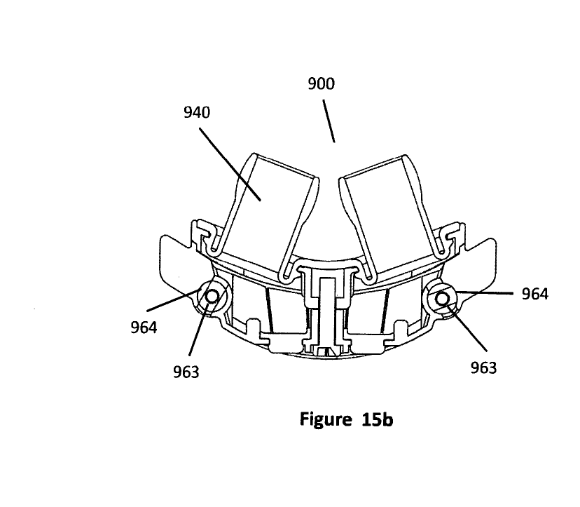

FIG. 15 depicts an exploded perspective view of another embodiment 900. The modulator 900 comprises a V-shaped valve 955. The valve conforms in shape to an interior wall of the modulator 900, which has vents 956 therealong. When suction (inhalation) is applied on nostril pieces 940, the valve 955 moves to freely allow air through the modulator, whereas when pressure (exhalation) is applied on the valve 955 towards the vents 956, the valve is moved to obstruct or restrict air exit via the vents 956. As shown, the valve 955 may be connected to the casing 954, but has flaps 957.

The modulator 900 further comprises swivel shutters 963, and static cages 964. As shown in the cross-section FIG. 15b, the shutters 963 may be individually controlled, so that airflow is limited to different extents through each. The shutter 963 on the left is aligned with the left cage 964 so that air may flow relatively unrestricted outwards, whereas the shutter 963 on the right is closed. The shutter 963 may easily be manipulated by use of a Phillips screwdriver. The shutters may be isolated from each other to allow independent control of breathing via each nostril.

Note that the nostril piece 940 is sufficiently wide to help seal and widen the nostril for more effectiveness. Typically they are made of soft silicon rubber.

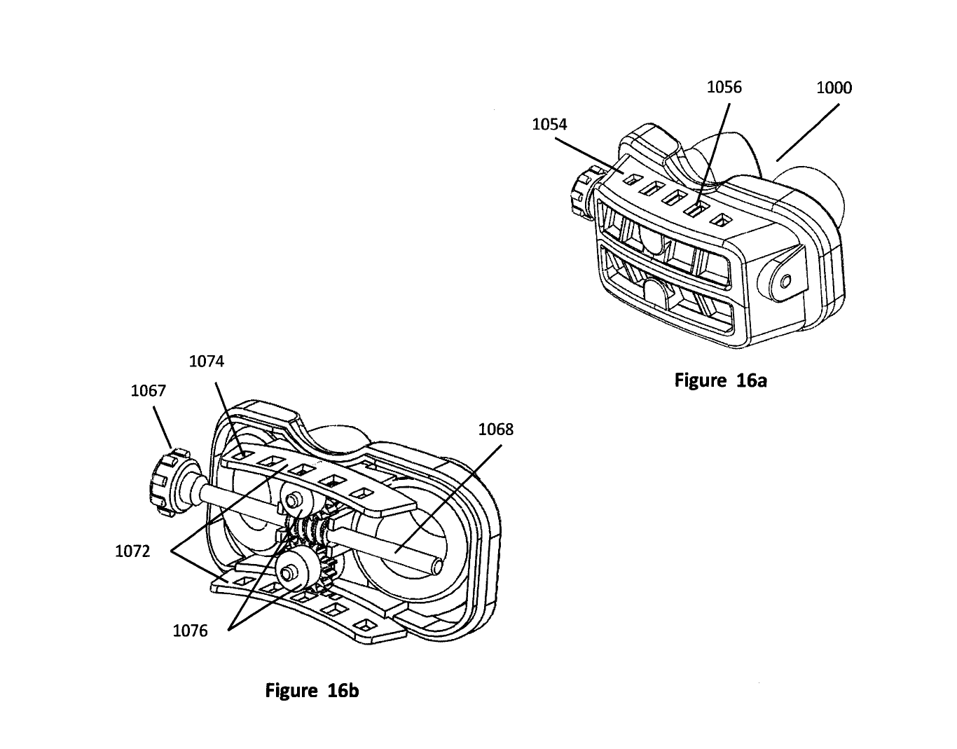

FIGS. 16a and 16b show another embodiment. The modulator 1000 again has vents 1056 on its casing 1054; a knob 1067 is operationally connected to a thread 1068, and the thread on 1068 is coupled to a shutter 1072 with slits 1074 via a gear 1076.

Manipulation of the knob 1067 leads to the movement of the shutter 1072 to the right and to the left. Alignment of the shutter slits 1074 with the vents 1056 accordingly is controlled and thus the resistance to exhalation may be controlled.

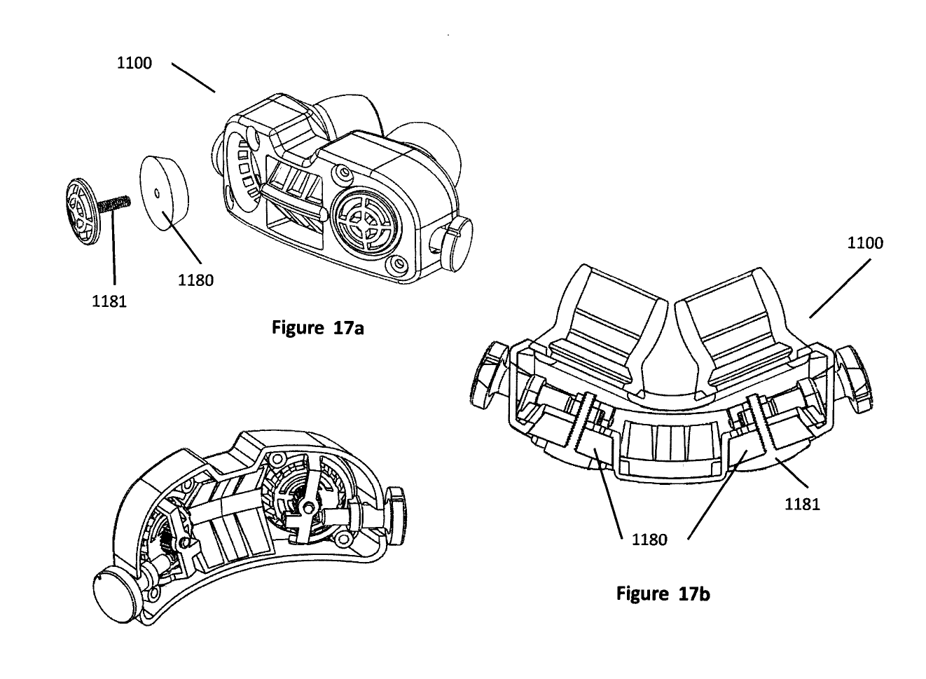

FIG. 17a illustrates in perspective views another modulator 1100 in which there is an article 1180 in each air passage allowing control of the resistance to exhalation. The article comprises pores 1181 that are blocked or distorted as a result of being squeezed by closing screws 1181. FIG. 17b depicts a cross section of the modulator 1100.

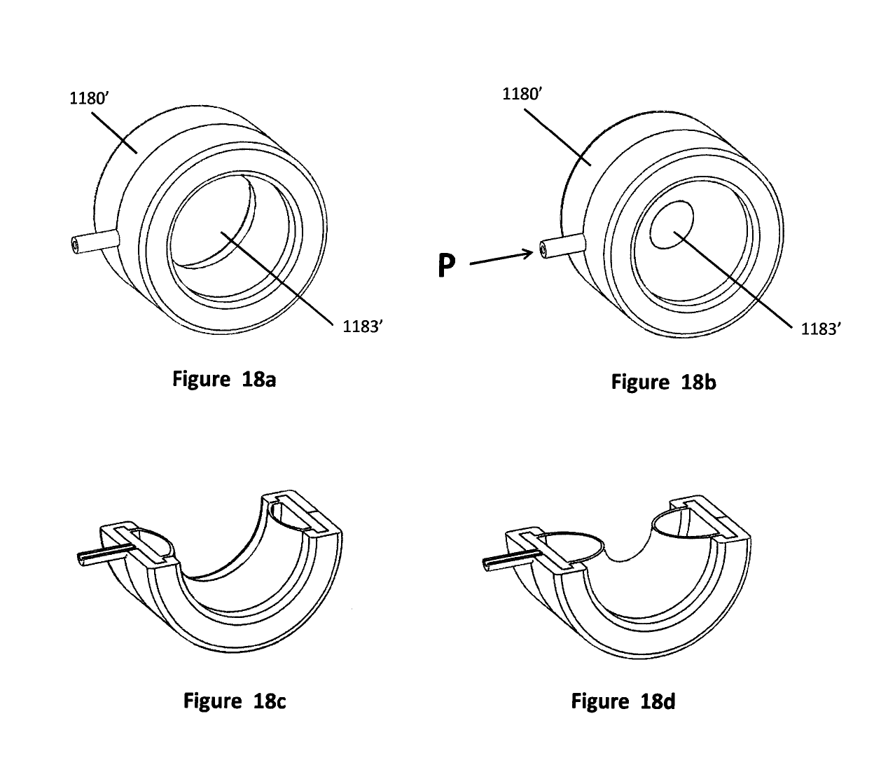

FIGS. 18a-d depict perspective views and cross-sections of another article 1180′, which is flexible, and which comprises an orifice 1183′. Applying hydraulic pressure to the article 1180′ leads to the orifice 1183′ diminishing in size, as shown in FIG. 18b relative to FIG. 18a. Typically, with embodiments having controllers, the exhalation resistance is increased as the individual drifts into deeper and deeper sleep. The resistance may be reset when the individual wakes up, either manually or automatically, and may further be modulated as the user drifts into deeper sleep. In some embodiments the resistances are adjustable to suit each user. As mentioned before, another important aspect is changing the resistance while the user gets used to the modulator.

The exterior surface of the article 1180′, in particular proximal to the orifice 1183′, may be very smooth. The smoothness may assist in reducing the noise the exhalation of air causes. The material from which at least this region of the article is made of may be specially selected for its smoothness. In addition, the contours of the surface in contact with air may be rounded.

An important aspect is the use of the modulator in various ways. In particular, alternating pressure on nasal cavities (in embodiments having two separately controlled valves) has found to be generally soothing, and is helpful in relaxation before falling asleep and adapting to the modulator. Thus another aspect is alternating operation of the valves, which helps train individuals in use of the modulator.

In some embodiments, the alternation of the resistance to exhalation via each nostril is also beneficial and thus the alternating pressure may be continued throughout the sleep process.

In other embodiments, the alternation is turned on and off according to the sleep state. The operation of the system may be customized to the preferences of the user and to the results of the various uses, which may have different effects for various users.

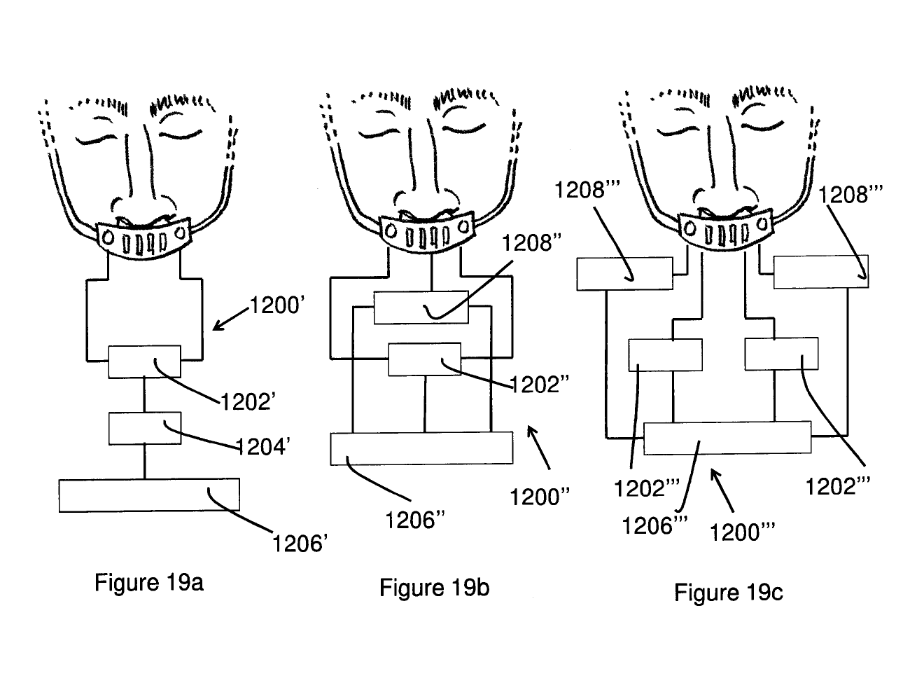

FIGS. 19a to 19c schematically present three systems 1200′, 1200″ and 1200′″ respectively that can modulate the breathing.

The first embodiment 1200′ comprises a motor 1202′, a timer 1204′ and a microprocessor 1206′. The second 1200″ comprises a motor 1202″, a microprocessor 1206″ and a sensor 1208″. The third 1200′″ comprises two motors 1202′″, a microprocessor 1206′ and two sensors 1208′″.

The microprocessors 1206′, 1206″, 1206′″ control the motors for closing/opening the air valves.

Typically when the user is falling asleep the system is entirely open both ways for free passage of air. As the user sleeps the air exit valves slowly increasingly close.

One option is that the operation is timed (system 1200′). Another is that the breath rate is monitored with an appropriate sensor 1208″. As the rate drops to a predetermined rate the system 1200″ kicks in. A system 1200′″ with two motors 1202′″ allows to separately control breathing through each nostril. The motors 1202′″ alternately operate the valves. The alternate operation may promote a calming or meditative state in the user. The system 1200′ may be configured to allow simultaneous and identical control of the valves when the sensors 1208′ sense a state of sleep according to the breath rate.

In some embodiments the nostril pieces are elongated and shaped to help support the nasal cavities, for individuals that have problems of restricted or “collapsing” nasal cavities.

It is stressed that no air supply is required by the embodiments described above, thus no tubing and external equipment is tied to the user, and no parts are adhesive, in contrast to some commercially available breathing aids. The embodiments described above are compact and easily carried around by the user.

However, further embodiments do comprise external pumps and tubing that allow breathing modulation, but include separate valves for each nostril to allow separate and individual control of breathing through each nostril.

Modulation of the resistance may be preprogrammed according to times for example, or may be dynamic and interactive according to measured parameters, for example the level of oxygen may be monitored, for example at a user's finger, using commercially available oxygen sensors operationally coupled to the computer directing the motor/s, and/or the breathing volume per time/air velocity may similarly be monitored to control the modulation.

The shapes and structures of the casings, valves etc may be changed without departing from the essence of the invention that includes improved regulation of air flow through nostrils.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A nasal breathing aid comprising the following components:
a housing;
facial engagement means attached thereto;
at least two nostril pieces coupled to the housing;
on the housing:
at least two nasal inlets;
at least one vent;
in the housing:
a first passage and a second passage, each communicating an inlet with a vent, and fluid-isolated from each other, and
restriction means configured to allow:
free flow of air through each passage from a vent to an inlet, and flow of air through the first passage independently restricted from flow of air through the second passage, from an inlet to a vent.

2. The aid of claim 1, further comprising means to alternately adjust restriction of air through the first passage and the second passage.

3. The aid of claim 1 or 2, further comprising means to adjust restriction of air through the first passage and the second passage to different extents.

4. A nasal breathing aid comprising the following components:
a housing;
facial engagement means attached thereto;
at least two nostril pieces coupled to the housing;
on the housing:
at least two nasal inlets;
at least one vent;
in the housing;
at least one passage communicating the inlets with the vents, and
restriction means configured to allow:
unimpeded flow of air through the passage from the vents to the inlets, and
restricted flow of air through the passage from the inlets to the vents, and
characterized by:
the breathing aid excluding skin-adhesive material;
the aid being wearable;
restriction means comprising:
membranes, each comprising an outer perimeter and an inner perimeter, the inner perimeter defining an orifice; wherein the outer perimeter is engaged between a nostril piece and the housing; and wherein the inner perimeter abuts the inlet when air is exhaled through the nostril pieces and does not abut the inlet, and the orifice expands towards the nostril pieces, when air is inhaled through the nostril pieces.

5. The breathing aid of claim 1, 2 or 4, further comprising at least one resilient valve, each valve conforming to an inlet such that below a predetermined exhale force the valve essentially blocks the inlet, and at a predetermined exhale force the valve unblocks the inlet.

6. The breathing aid of claim 5, further comprising an actuator configured to allow adjusting a force holding the valve against the inlet.

7. The breathing aid of claim 6, further comprising:
an elastic strap coupled to the housing;
electric wiring within or on the strap;
automatic control means programmed to control the actuator, wherein the electrical wiring electrically connects the automatic control means with the actuator.

8. The breathing aid of claim 1, 2 or 4, further comprising swivel shutters, static cages and at least one screw drive, wherein the screw drive is coupled to the swivel shutters, and the swivel shutters are arranged relative to the cages such that rotation of the screw drives rotates at least one shutter, to adjust gaps between the cages and the shutters.

9. The breathing aid of claim 8, further comprising at least one dial protruding from the housing, the dials coupled to the screw drives such that manipulation of a dial leads to rotation of at least one shutter.

10. The breathing aid of claim 1, 2 or 4, further comprising easily replaceable mucus-absorbent pads.

11. The breathing aid of claim 10, wherein the pads are disposed in the nostril pieces.

12. The breathing aid of claim 10, wherein the pads are disposed in the passages.

13. The breathing aid of claim 10, wherein each pad comprises:
an essentially flat layer of mucus-absorbent material;
ribs attached to the layer,
and a handle extending from the ribs.

14. The breathing aid of claim 5, wherein the valves are spring loaded.

15. The breathing aid of claim 5, wherein the valve comprises an elastomeric element proximal to the inlet such compression of the element impedes flow of air through the passages.

16. The breathing aid of claim 1, 2 or 4, further comprising at least one air pump and tubing coupling the pump to the housing.

17. The breathing aid of claim 16, wherein the aid is configured to allow the pump to alternately supply air to each nasal inlet.
